(19) United States
(12) Patent Application Publication
(10) Pub. No.: US 2005/0245785 A1
(43) Pub. Date: Nov. 3, 2005

(54) Biodegradable seed placement device and method

(76) Inventors: Theodore T. Tarone, West Palm Beach, FL (US); Mario LaCasse, Palm Beach Gardens, FL (US); Daniel Haber, Wellington, FL (US)

Correspondence Address: STROOCK & STROOCK & LAVAN LLP, 180 MAIDEN LANE, NEW YORK, NY, 10038, US

(21) Appl. No.: 10/975,746

(22) Filed: Oct. 28, 2004

Related U.S. Application Data
(60) Provisional application No. 60/515,492, filed on Oct. 29, 2003.

(51) Int. Cl. A61N 5/00; A61M 36/00

(52) U.S. Cl. 600/7

(57) ABSTRACT

A biodegradable radioactive seed placement system is provided comprising a thin, flexible, biodegradable sheath having at least one radioactive isotope seed positioned between a first end and a second end. Additionally, at least one spacer can be positioned between the first end and the second end adjacent to a radioactive isotope seed. The sheath/seed assembly can be loaded into a needle and implanted from the needle into a patient.

### RELATED U.S. PATENT APPLICATIONS

This application claims the benefit of U.S. Provisional Application No. 60/515,492, filed Oct. 29, 2003, which is incorporated herein by reference in its entirety.

### TECHNICAL FIELD

The present invention relates to radioactive implants for medical therapeutic purposes, referred to in the art as “radioactive seeds,” “seeds,” or “sources” for therapeutic radiation treatment of oncological and other medical conditions. More particularly, the present invention is directed to a novel biodegradable radioactive seed placement device for interstitial implantation brachytherapy and also for general brachytherapy treatments. The invention is also directed to methods of making the biodegradable radioactive seed placement device and methods of using the same.

### BACKGROUND OF THE INVENTION

The localized treatment of tumors and other medical conditions by the interstitial implantation of radioactive materials is a recognized treatment modality of long standing. Radioactive implants are used to provide radiation therapy in order to reduce or prevent the growth of tumors that cannot be removed by surgical means. Radioactive implants are also used to prevent the growth of microscopic metastatic deposits in lymph nodes that drain the region where a tumor has been removed. Implants are also used to irradiate the postoperative tumor bed after the tumor is excised. Implantation of radioactive sources directly into solid tumors for the destruction of the tumors is used in a therapy referred to as brachytherapy.

Brachytherapy is also used to prevent the regrowth of tissue in circumstances such as the treatment of arteries for occlusive disease. Brachytherapy is applied, for example, in the treatment of atherosclerosis to inhibit restenosis of blood vessels after balloon-angioplasty or other treatments to open occluded or narrowed vessels. These brachytherapy treatments involve a short-term application of extremely radioactive sources. The applications can be for periods as short as a few minutes. This form of brachytherapy may therefore be contrasted with the treatment of tumors where lower activity sources are used for longer periods of time that may be measured in hours or days or may involve permanent implantation.

Treatment of medical conditions with the local application of radiation by implantation concentrates the treatment on the adjacent tissue and advantageously minimizes the exposure of more distant tissues that it is not desired to irradiate. Direct implantation of radioactive sources into tumors often permits the application of larger doses of radiation than may otherwise be achieved because the radiation is applied directly at the site to be irradiated. Local application of brachytherapy to non-cancerous conditions also allows the use of more intensive treatment than is possible by other means.

In the prior art, brachytherapy “sources” are generally implanted for short periods of time and usually are sources of high radiation intensity. For example, irradiation of body cavities such as the uterus has been achieved by placing radium-226 capsules or cesium-137 capsules in the lumen of the organ. In another example, tumors have been treated by the surgical insertion of radium needles or iridium-192 ribbons into the body of the tumor. In yet other instances gold-198 or radon-222 have been used as radioactive sources. These isotopes require careful handling because they emit highly energetic and penetrating radiation that can cause significant exposure to medical personnel and to the normal tissues of the patient undergoing therapy. Therapy with sources of this type requires that hospitals build shielded rooms, provide medical personnel with appropriate protection and establish protocols to manage the radiation hazards.

The prior art interstitial brachytherapy treatment using needles or ribbons has features that inevitably irradiate normal tissues. For example, normal tissue surrounding the tumor is irradiated when a high energy isotope is used even though the radiation dose falls as the square of the distance from the source. Brachytherapy with devices that utilize radium-226, cesium-137 or iridium-192 is hazardous to both the patient and the medical personnel involved because of the high energy of the radioactive emissions. The implanted radioactive objects can only be left in place temporarily; thus the patient must undergo both an implantation and removal procedure. Medical personnel are thus twice exposed to a radiation hazard.

In prior art brachytherapy that uses long-term or permanent implantation, the radioactive device is usually referred to as a “seed.” Where the radiation seed is implanted directly into the diseased tissue, the form of therapy is referred to as interstitial brachytherapy. It may be distinguished from intracavitary therapy, where the radiation seed or source is arranged in a suitable applicator to irradiate the walls of a body cavity from the lumen.

Migration of the device away from the site of implantation is a problem sometimes encountered with presently available iodine-125 and palladium-103 permanently implanted brachytherapy devices because no means of affirmatively localizing the device may be available.

The prior art discloses iodine seeds that can be temporarily or permanently implanted. The iodine seeds disclosed in the prior art consist of the radionucleide adsorbed onto a carrier that is enclosed within a welded metal tube. Seeds of this type are relatively small and usually a large number of them are implanted in the human body to achieve a therapeutic effect. Individual seeds of this kind described in the prior art also intrinsically produce an inhomogeneous radiation field due to the form of the construction.

The prior art also discloses sources constructed by enclosing iridium metal in plastic tubing. These sources are then temporarily implanted into accessible tissues for time periods of hours or days. These sources must be removed and, as a consequence, their application is limited to readily accessible body sites.

Prior art seeds typically are formed in a manner that differs from isotope to isotope. The form of the prior art seeds is thus tailored to the particular characteristics of the isotope to be used. Therefore, a particular type of prior art seed provides radiation only in the narrow range of energies available from the particular isotope used.

Brachytherapy seed sources are disclosed in, for example, U.S. Pat. No. 5,405,165 to Carden, U.S. Pat. No. 5,354,257 to Roubin, U.S. Pat. No. 5,342,283 to Good, U.S. Pat. No. 4,891,165 to Suthanthirian, U.S. Pat. No. 4,702,228 to Russell et al, U.S. Pat. No. 4,323,055 to Kubiatowicz and U.S. Pat. No. 3,351,049 to Lawrence, the disclosures of which are incorporated herein by reference.

The brachytherapy seed source disclosed by Carden comprises small cylinders or pellets on which palladium-103 compounded with non-radioactive palladium has been applied by electroplating. Addition of palladium to palladium-103 permits electroplating to be achieved and allows adjustment of the total activity of the resulting seed. The pellets are placed inside a titanium tube, both ends of which are sealed. The disclosed invention does not provide means to fix the seed source within the tissues of the patient to ensure that the radiation is correctly delivered. The design of the seed source is such that the source produces an asymmetrical radiation field due to the radioactive material being located only on the pellets. The patent also discloses the use of end caps to seal the tube and the presence of a radiographically detectable marker inside the tube between the pellets.

The patent to Roubin relates to radioactive iridium metal brachytherapy devices positioned at the end of minimally invasive intravascular medical devices for providing radiation treatment in a body cavity. Flexible elongated members are disclosed that can be inserted through catheters to reach sites where radiation treatment is desired and that can be reached via vessels of the body.

The patent to Good discloses methods such as sputtering for applying radioactive metals to solid manufactured elements such as microspheres, wires and ribbons. The disclosed methods are also disclosed to apply protective layers and identification layers. Also disclosed are the resulting solid, multilayered, seamless elements that can be implanted individually or combined in intracavitary application devices.

The patent to Suthanthirian relates to the production of brachytherapy seed sources and discloses a technique for use in the production of such sources. The patent discloses an encapsulation technique employing two or more interfitting sleeves with closed bottom portions. The open end portion of one sleeve is designed to accept the open end portion of a second slightly-smaller-diameter sleeve. The patent discloses the formation of a sealed source by sliding two sleeves together. Seeds formed by the Suthanthirian process may have a more uniform radiation field than the seed disclosed by Carden. However, the seed disclosed by Suthanthirian provides no means for securely locating the seed in the tissue of the patient.

The patent to Russell et al. relates to the production of brachytherapy seed sources produced by the transmutation of isotopically enriched palladium-102 to palladium-103 by neutrons produced by a nuclear reactor. The Russell patent also discloses a titanium seed with sealed ends, similar to that of Carden, containing a multiplicity of components. A seed produced in this manner is associated with yielding a less than isotropic radiation field.

The patent to Kubiatowicz teaches a titanium seed with ends sealed by laser, electron beam or tungsten inert gas welding. The radioactive component of the seed is disclosed to be a silver bar onto which the radioisotope iodine-125 is chemisorbed. Seeds produced in this manner also tend to produce an asymmetric radiation field and provide no means of attachment to the site of application in the patient.

The patent to Lawrence discloses a radioactive seed with a titanium or plastic shell with sealed ends. Seeds are disclosed containing a variety of cylindrical or pellet components onto which one of the radioisotopes iodine-125, palladium-103 or cesium-131 is incorporated. The structure of the disclosed seeds yields a non-homogeneous radiation field and provides no means for accurately positioning the seed in the tissue that it is desired to irradiate.

Currently available brachytherapy seeds do not easily lend themselves to association with suture material. For example, iodine-125 seeds currently in use are placed inside suture material at the time of manufacture. However, the insertion process is tedious and time consuming and has the potential for significant radiation exposure to the production personnel involved. Additionally, because of the natural decay of the radioisotope, the suture material thus produced has a short shelf life. As a second example, the manufacturing process used to produce the palladium-103 seeds that are currently in use results in end-roughness of the encapsulation of the seed. The capsules are not placed inside suture material because the end-roughness makes insertion very difficult. Rigid rods are produced in present technology by the insertion of seeds into suture material followed by heat treatment to form a rigid rod containing the seed. These rods are difficult to produce, very fragile and sensitive to moisture. The presently available brachytherapy technology requires that most physicians use suture material preassembled with the seeds already inside. Similarly, rigid materials used by surgeons for brachytherapy are pre-manufactured and purchased readymade.

Thus, there is a need for a biodegradable radioactive seed placement device and a method of making a biodegradable radioactive seed placement device that overcomes the aforementioned shortcomings of the prior art by providing better accuracy for seed placement, simplifying implantation procedures, permitting seeds to be loaded in multiple configurations, and precluding seed migration.

### SUMMARY OF THE INVENTION

The present invention eliminates the above-mentioned needs for a biodegradable radioactive seed placement device by providing a biodegradable radioactive seed placement device and a method of producing a biodegradable radioactive seed placement device that provides better accuracy for seed placement, simplifies implantation procedures, permits seeds to be loaded in multiple configurations, and precludes seed migration.

In accordance with the present invention, there is provided a biodegradable radioactive seed placement device including a biodegradable sheath having a first end and a second end, and at least one radioactive isotope seed positioned between the first end and the second end. Additionally, at least one spacer can be positioned between the first end and the second end and adjacent to the at least one radioactive isotope seed.

The present invention is further directed to a method for loading a needle with a radioactive isotope, the method including the steps of placing a biodegradable sheath incorporating at least on radioactive isotope seed in a first position within a housing having a needle attachment end and a loading end, positioning a needle in an operative engagement with the needle attachment end, and, transferring the biodegradable sheath incorporating at least on radioactive isotope seed from the first position in the housing to a second position in the needle

### BRIEF DESCRIPTION OF THE DRAWINGS

FIG. 1 is a cross-sectional view of the preferred embodiment of the present invention.

FIG. 2 is a cross-sectional view of an alternative embodiment of the present invention of FIG. 1.

FIG. 3 is a cross-sectional view of the alternative embodiment of FIG. 2 used in accordance with the preferred method of the present invention.

FIG. 4 is a cross-sectional view of the present invention of FIG. 3 illustrating a technique for loading a needle.

FIG. 5 is a cross-sectional view of the present invention of FIG. 4 illustrating a release from the needle.

### DETAILED DESCRIPTION OF THE DRAWINGS

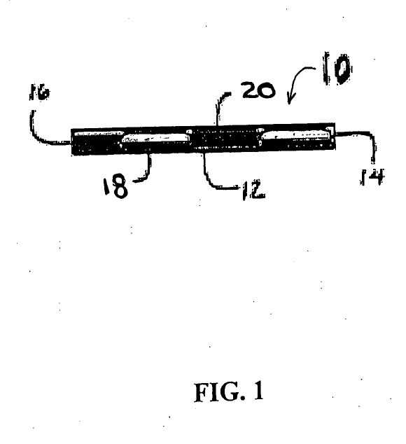

Referring now to FIG. 1, a preferred embodiment of the present invention is illustrated as biodegradable seed placement device 10. Biodegradable seed placement device 10 includes a biodegradable sheath 12, a first end 14, a second end 16, and at least one radioactive isotope seed 18 positioned between the first end 14 and the second end 16. Additionally, at least one spacer 20 may be positioned between the first end and the second end and adjacent to the at least one radioactive isotope seed 18.

Once inserted into the diseased tissue of a patient, biodegradable seed placement device 10, through the incorporation of biodegradable sheath 12, prevents seeds 18 and spacers 20 from migrating out of position and into undesired locations within the patient. Migration of seeds 18 and spacers 20 is prevented by the size of biodegradable seed placement device 10 that is implanted into the patient. Alternatively, after a sufficient period of time, biodegradable seed placement device 10 can become more fixed in its position as a result of the patient's body immune response to the presence of biodegradable seed placement device 10. As biodegradable seed placement device 10 becomes more fixed in its position, biodegradable sheath 12 begins to deteriorate and can be absorbed into the patient's body. Once biodegradable sheath 12 has deteriorated to the point where it can no longer contain seeds 18 and/or spacers 20, the patient's immune response will have created a situation where seeds 18 and/or spacers 20 are unable to migrate (such as from blockage by scar tissue) or migration is no longer relevant due to decreased radioactivity of seeds 18.

Biodegradable sheath 12 can be made from any one of a number of materials, including but not limited to biodegradable polymers, cellulose, lipids, and proteins.

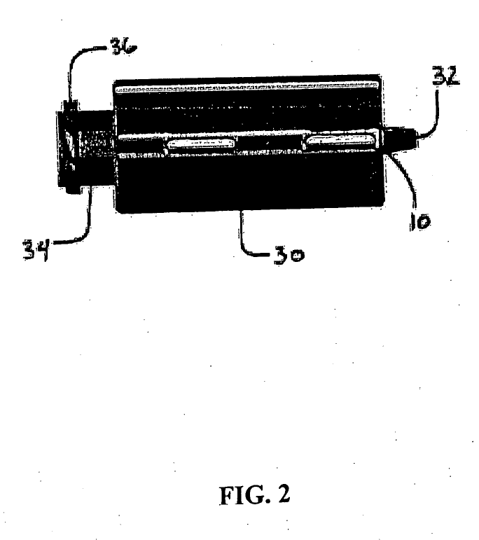

Referring now to FIG. 2, biodegradable seed placement device 10 is placed within a first position within a housing 30. Housing 30 includes a needle attachment end 32 and a loading end 34. Loading end 34 includes attachment 36 for adapting housing 30 to fit a variety of devices, such as a semi-automatic needle loader or any one of a number of traditional needle loading systems. Additionally, housing 30 is constructed of materials or in manners that assist in the reduction or prevention of the passage of radioactive energies from seeds 18 to the outside environment.

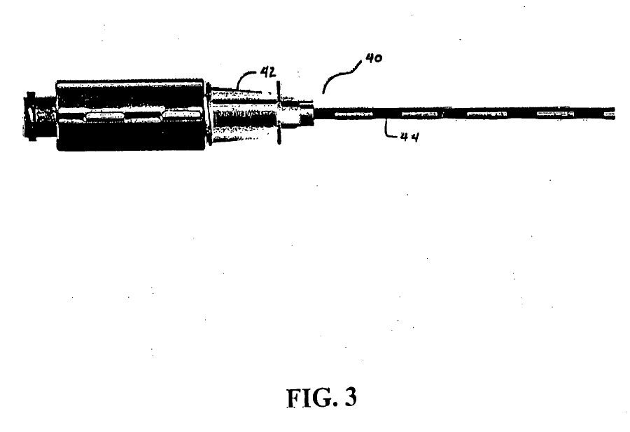

As illustrated in FIG. 3, a needle group 40 can be loaded onto housing 30 at needle attachment end 32. As is shown, needle attachment 42 is placed over needle attachment end 32 so that a needle 44 is positioned in an operative engagement with needle attachment end 32. The operative engagement between needle attachment end 32 and needle 44 permits the passage of biodegradable seed placement device 10 from housing 30 to needle 44.

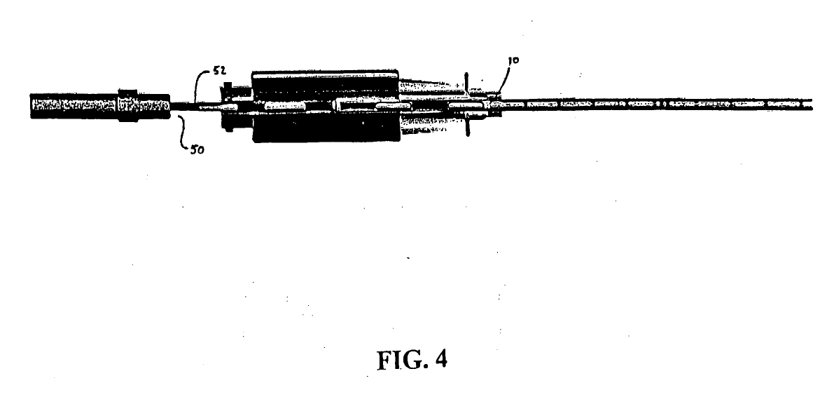

Referring now to FIG. 4, a stylet 50 is used to transfer biodegradable seed placement device 10 from its first position within housing 30 to a second position within needle 44. In order to accomplish this repositioning, push rod 52 of stylet 50 must have a diameter that is greater than the diameter of the inner portion of biodegradable sheath 12 to prevent push rod 52 from pushing seeds 18 and/or spacers 20 out of biodegradable sheath 12. Once biodegradable seed placement device 10 is transferred into needle 44, needle 44 is disengaged from needle attachment end 32 of housing 30 and prepared for insertion into the patient.

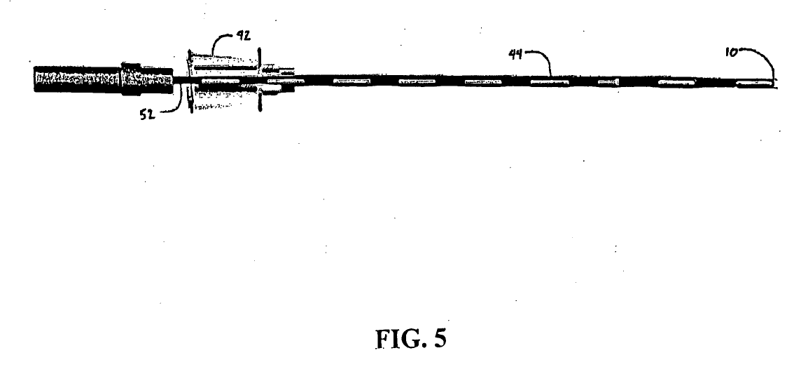

In order to transfer biodegradable seed placement device 10 from needle 44 to the tissue of a patient, push rod 52 is employed. As shown in FIG. 5, push rod 52 fits within needle 44 and drives biodegradable seed placement device 10 from its position within needle 44 to a position within the patient. As stated above, in order to accomplish the positioning of biodegradable seed placement device 10 within the patient, push rod 52 must have a diameter that is greater than the diameter of the inner portion of biodegradable sheath 12 to prevent push rod 52 from pushing seeds 18 and/or spacers 20 out of biodegradable sheath 12.

Although only a few exemplary embodiments of the present invention have been described in detail above and in the Figures below, those skilled in the art will readily appreciate that numerous modifications are to the exemplary embodiments are possible without materially departing from the novel teachings and advantages of this invention. Accordingly, all such modifications are intended to be included within the scope of this invention as defined in the following claims.

1. A biodegradable radioactive seed placement system, comprising:
a thin, flexible, biodegradable sheath having a first end and a second end; and
at least one radioactive isotope seed positioned between said first end and said second end.

2. The system of claim 1, comprising a plurality of seeds and spacers, wherein at least one spacer is positioned between said first end and said second end and adjacent to said at least one radioactive isotope seed.

3. The system of claim 1, wherein said sheath comprises biodegradable polymers.

4. The system of claim 1, wherein said sheath comprises cellulose.

5. The system of claim 1, wherein said sheath comprises lipids.

6. The system of claim 1, wherein said sheath comprises proteins.

7. The system of claim 1, wherein an inner diameter of said sheath is radially expanded when a seed and/or a spacer is inserted therein.

8. A biodegradable radioactive seed placement system, comprising:
a housing having a needle attachment end and a loading end;
a delivery chamber for placement of a thin, flexible, biodegradable sheath having a first end and a second end within said housing; and
at least one radioactive isotope seed positioned between said first end and said second end.

9. The system of claim 8, comprising a plurality of seeds and spacers, wherein at least one spacer is positioned between said first end and said second end and adjacent to said at least one radioactive isotope seed.

10. The system of claim 8, wherein the sheath is formed of resilient material.

11. A method for loading a needle with radioactive isotope, said method comprising:
placing a thin, flexible, biodegradable sheath incorporating at least one radioactive isotope seed in a first position within a housing having a needle attachment end and a loading end;
positioning a needle in an operative engagement with said needle attachment end; and
transferring said biodegradable sheath incorporating at least one radioactive isotope seed from said first position in said housing to a second position in said needle.
